(19) United States
(12) Patent Application Publication
(10) Pub. No.: US 2010/0036425 A1
(43) Pub. Date: Feb. 11, 2010

(54) ANTI-TORSION SPINE FIXATION DEVICE

(75) Inventors: Michael Barrus, Ashburn, VA (US); Kevin R. Strauss, Columbia, MD (US)

Correspondence Address: CARTER, DELUCA, FARRELL & SCHMIDT, LLP, 445 BROAD HOLLOW ROAD, SUITE 420, MELVILLE, NY, 11747, US

(73) Assignee: K2M, Inc., Leesburg, VA (US)

(21) Appl. No.: 12/536,602

(22) Filed: Aug. 6, 2009

Related U.S. Application Data
(60) Provisional application No. 61/188,090, filed on Aug. 6, 2008.

(51) Int. Cl. A61B 17/70; A61B 17/86

(52) U.S. Cl. 606/264; 606/301

(57) ABSTRACT

An anti-torsion spine fixation device includes an elongated member spanning from one vertebra to another and connected to each vertebra. The anti-torsion spine fixation device may span more than one vertebral level, but is fixed bilaterally to the most cephalad and caudad vertebrae.

### CROSS REFERENCE TO RELATED APPLICATIONS

This application claims priority to, and the benefit of, U.S. Provisional Patent Application Ser. No. 61/188,090 filed Aug. 6, 2008. The entire contents of the aforementioned application are incorporated by reference herein.

### TECHNICAL FIELD

The present disclosure relates to orthopedic spine surgery, and more particularly, to apparatuses and methods for stabilizing and fixing the spine.

### BACKGROUND AND RELATED ART

Correction of a spinal deformity typically requires stabilization and fixation of vertebrae in a particular spatial relationship. Surgical spinal correction procedures involve the placement of a plurality of bone pins, anchors, cables, hooks, or screws placed in adjacent vertebrae and using spinal rods to maintain a predetermined spatial relationship between the vertebrae. Such devices may be permanently implanted in the subject. However, in other cases, the devices may be subsequently removed when no longer needed.

In an effort to maintain normal growth or height while correcting a younger patient's abnormally curved spine, unilateral constructs may be implanted with the purpose of maintaining height on one side of the spine, the convex side of the curve, while the concave side continues to grow. Over time, this method of instrumentation may, on the concave side of the scoliotic curve, grow the spine straight.

Spinal instrumentation such as pedicle screws and rods may be used to achieve this type of correction. Some traditional rod and screw constructs are subject to becoming misaligned over time.

### SUMMARY

An anti-torsion spine fixation device includes a plurality of anchors disposed on opposing pedicles of at least two vertebrae disposed adjacent to a scoliotic curve with a connecting rod traversing the anchors such that the path of the rod approximates a “C”. The anchors closest to the convex portion of the scoliotic curve are coupled by the rod. There is no corresponding coupling structure near the concave portion of the scoliotic curve, thereby defining a gap or “corrective opening” in the rod's path which corresponds to the concave portion of the scoliotic curve.

The anti-torsion spine fixation device so configured allows for corrective growth at the corrective opening while restricting growth near the convex portion of the scoliotic curve. Additionally, the anti-torsion spine fixation device inhibits further rotation of a non-scoliotic spine. Further, because the rod is joined to opposing anchors on a single vertebra, the anti-torsion spine fixation device limits torsional motion of the spine by requiring the torsional motion to be acted on the uni-lateral length of rod which is fixed to bilateral anchors of at least one vertebra.

According to another aspect of the present disclosure, an anti-torsion spine fixation device includes a plurality of anchors and rod segments coupled to vertebrae configured to define multiple opposing corrective openings. The path of the correcting rod is configured such that the device both allows growth at each corrective opening and restricts torsion along its length.

According to another aspect of the present disclosure, rod segments may be retained in each anchor by a setscrew. According to another aspect of the present disclosure, rod segments may be retained in each anchor by a clamp. According to another aspect of the present disclosure, anchors may be secured to their respective locations upon a vertebra by a pedicle screw.

### BRIEF DESCRIPTION OF THE DRAWINGS

The foregoing and other features of the present disclosure will become apparent to one skilled in the art to which the present disclosure relates upon consideration of the following description of the disclosure with reference to the accompanying drawings, wherein:

FIG. 1 is an anterior plan view of an anti-torsion spine fixation device coupled to two vertebrae;

FIG. 2 is a perspective view of an anti-torsion spine fixation device of FIG. 1;

FIG. 3 is an anterior plan view of the anti-torsion spine fixation device configured in a bi-directional construct;

FIG. 4 is an isometric view of the anti-torsion spine fixation device of FIG. 3;

FIG. 5 is an anterior plan view of an anti-torsion spine fixation device having an expanding member configured to accommodate growth of a patient;

FIG. 6 is a perspective view of the anti-torsion spine fixation device of FIG. 5;

FIG. 7a is a perspective view of a first polyaxial bone screw; and

FIG. 7b is a perspective view of a second polyaxial bone screw.

### DETAILED DESCRIPTION OF THE EMBODIMENTS

Embodiments of the presently disclosed apparatuses and methods for spinal surgery will now be described in detail with reference to the appended drawings, in which like reference numerals designate identical or corresponding elements in each of the several views. Throughout the following description, the term “proximal,” will refer to the end of a device or system that is closest to the operator, while the term “distal” will refer to the end of the device or system that is farthest from the operator. In addition, the “long axis of the spine” runs approximately in the direction from the head to the tailbone, with the direction toward the head referred to as being “cephalad” and the direction toward the tailbone referred to as being “caudad.” Further still, for the purposes of this application, the term “medial” indicates a direction toward the middle of the body of the patient while the term “lateral” indicates a direction away from the middle of the body of the patient.

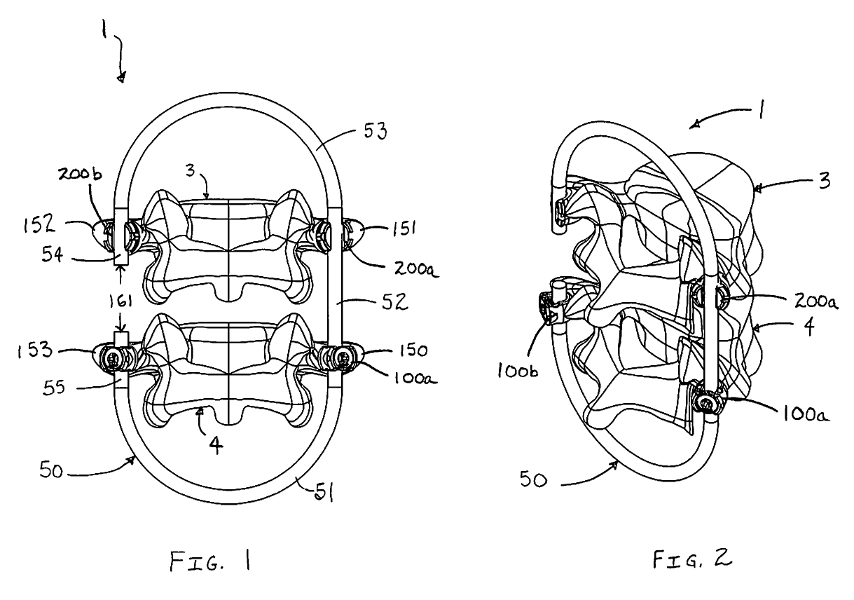

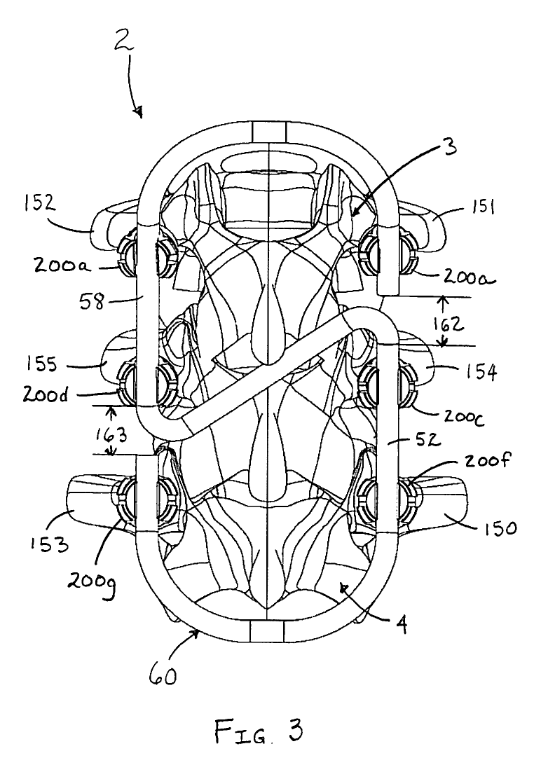

A spinal fixation device 1 will now be described with reference to FIGS. 1 and 2. The spinal fixation device 1 includes a rod 50, having rod segments 51, 52, 53, 54, and 55. Rod 50 is coupled to anchors 100a, 200a, 200b, and 100b. Although rod 50 is illustrated as including a plurality of rod segments for ease of explaining the disclosed features, it is contemplated that rod 50 may be a single continuous rod that is shaped to fit the desired anchor locations or may be a number of rod segments coupled together that form rod 50. Anchors 100a and 100b arc coupled to caudad vertebra 4 at respective locations 150 and 153. Anchors 200a, and 200a are coupled to cephalad vertebrae 3 at respective locations 151 and 152.

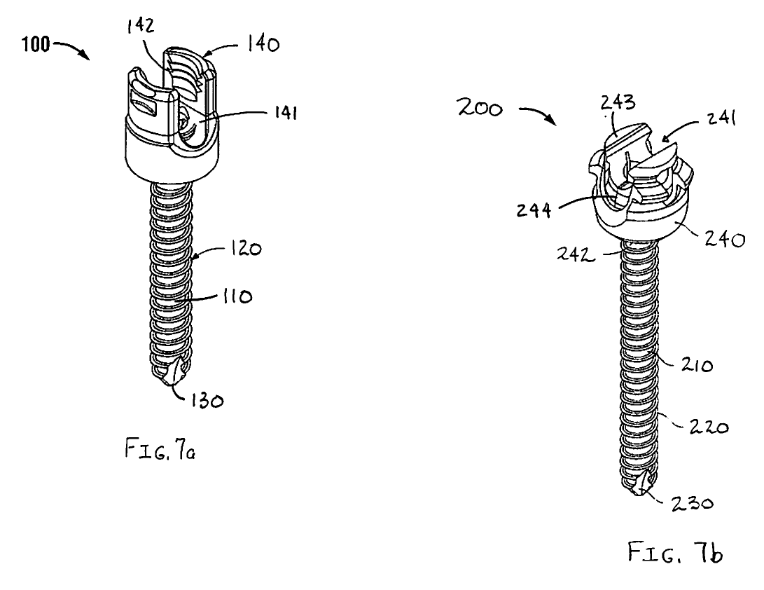

In the present disclosure, the term “anchor” refers to devices suitable for coupling one or more rods to one or more bone structures such as a vertebral body. For example, with reference to FIG. 1, anchors 200a and 200b disposed on cephalad vertebrae 3 are shown as taper lock style polyaxial screws. One example of a taper lock style polyaxial screw is disclosed in commonly assigned International Patent Application Publication No. PCT/US2008180682, filed on Oct. 22, 2008, and shown in FIG. 7b of the present disclosure as anchor 200. Similarly, anchors 100a and 100b disposed on caudad vertebrae 4 are shown as polyaxial style screws such as those disclosed in commonly assigned International Patent Application Publication No. PCT/US2008/80668, filed on Oct. 22, 2008, and shown in FIG. 7a of the present disclosure as anchor 100. Both aforementioned applications are incorporated by reference herein in their entirety. Either of these bone anchor types may be used to couple portions of rod 50 to vertebrae.

With reference to FIG. 7a, a bone anchor 100 is shown having, an elongated shaft 110 defining a longitudinal axis having a distal end portion and a proximal end portion, a helical thread 120 disposed thereupon, a substantially conical distal tip 130, and a proximal head assembly 140. Proximal head assembly 140 and elongated shaft 110 are pivotably coupled to allow angular displacement of proximal head assembly 140 relative to the longitudinal axis. Further, proximal head assembly 140 has a generally U-shaped cross-section defining a channel 141 configured to retain a rod such as spinal fixation rod 50 shown in FIG. 1. Further still, there are opposing threads 142 disposed on opposing faces of channel 141 configured to receive a set-screw (not shown) capable of retaining a rod.

With reference to FIG. 7b, a bone anchor 200 is shown having, an elongated shaft 210 defining a longitudinal axis having a distal end portion and a proximal end portion, a helical thread 220 disposed thereupon, a substantially conical distal tip 230, and a proximal head assembly 240. Proximal head assembly 240 and elongated shaft 210 are pivotably coupled to allow angular displacement of proximal head assembly 240 relative to the longitudinal axis.

Further, proximal head assembly 240 includes a collet member 242 and a saddle member 243. Saddle member 243 has a generally U-shaped cross-section defining a channel 241. Further still, saddle member 243 has a slot 244 extending from the nadir of the channel 241 towards the bottom of saddle member 243 which essentially bisects the saddle member 241 along a central axis. It is contemplated that slot 244 may not extend all the way through the body portion. Proximal head assembly 240 is configured to retain a rod within channel 241 by the reducing the width of slot 244.

With reference to FIGS. 1 and 2, spinal fixation device 1 is configured to be disposed upon a patient's spine such that the convex portion of a scoliotic curve corresponds to rod segment 52 which spans anchors 100a and 200a while the concave portion of the scoliotic curve disposed between anchors 200b and 100b has no such corresponding connecting structure defining corrective gap 161 therebetween. Alternatively, the presently disclosed spinal fixation device 1 is adaptable for use in a patient where a uni-lateral rod is desired and the possibility of “crankshafting” is a concern. As a patient's spine grows, such an arrangement of rod segments and anchors allows the concave portion of the curve disposed within corrective gap 161 to grow while maintaining a substantially constant distance at the convex portion of the curve between anchors 100a and 200a, thereby helping to correct the scoliotic deformity.

Traditional unilateral spinal constructs may require additional stabilization to prevent or inhibit torsion about the long axis of the spine in addition to correction of the convex and concave portions of the scoliotic curve.

As shown in FIGS. 1 and 2, a rod segment 53 disposed on the cephalad vertebrae 3 approximates an arcuate path from rod segment 52 to 54 such that the apex of the arc is directed towards the patient's head. Similarly, a rod segment 51 disposed on caudad vertebrae 4 approximates an arcuate path from rod segment 52 to rod segment 55 such that the apex of the arc is directed towards the patient's feet.

Segments 51 and 53 provide additional coupling between vertebrae 4 and 3 beyond the clamping pressure exerted on segment 52 at anchors 100a and 200a. In such a configuration, rotation of anchors 100a and 200a relative to one another about the long axis of the spine is impossible without a corresponding translation of anchors 200b and 100b and consequently, a deformation of the rod segments between those anchors. Therefore, the resistance to torsional deformation of the anti-torsion spine fixation device may be defined by the torsional yield strength of the material from which the rod segments are made.

Where multiple scoliotic curves are present, additional anchors and rods may be configured in a curve which approximates multiple anti-torsion spine fixation devices whose corrective action is directed toward the multiple scoliotic curves while maintaining torsional rigidity about the long axis of the spine.

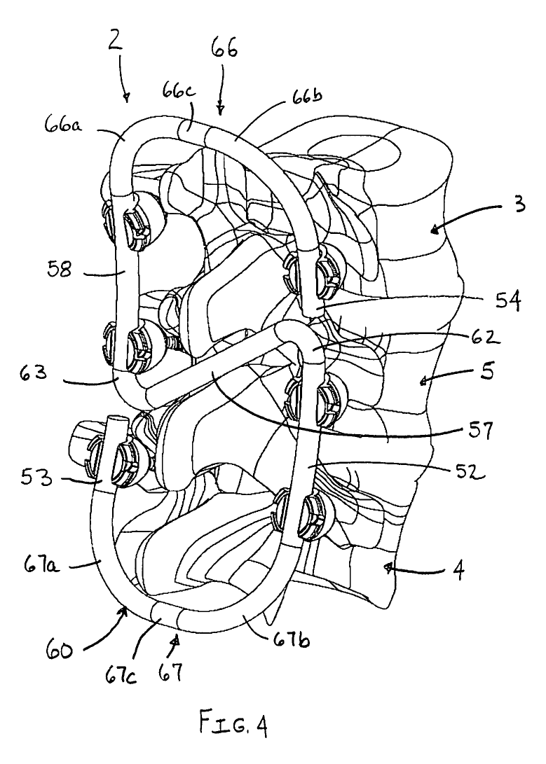

As shown in FIGS. 3 and 4, bi-lateral spinal fixation device 2 includes the constructs present in spinal fixation device 1 with the addition of constructs coupled to intermediate vertebrae 5 optionally disposed between cephalad vertebrae 3 and caudad vertebrae 4. The constructs disposed on vertebrae 3 and 4 are shown in FIGS. 1 and 2 and described hereinabove. The differences between spinal fixation device 1 and spinal fixation device 2 are described hereinbelow.

In this configuration, spine fixation device 2 has opposing anchors 200c and 200d at locations 154 and 155 on an intermediate vertebra 4. Additionally, spine fixation device 2 includes rod 60, which includes the rod segments present in rod 50 described hereinabove with the additional rod segments being discussed hereinafter. Rod segment 52 joins the cephalad portion of anchor 200f to the caudad portion of anchor 200c, rod segment 57 joins cephalad portion of anchor 200c to the caudad portion of anchor 200d, and rod segment 58 joins the cephalad portion of anchor 200d to the caudad portion of anchor 200b such that the curve approximated by adjoining rod segments defines opposing corrective gaps 162 and 163. Rod segments 52, 58 maintain the torsional rigidity of the device established by the curved paths of rods 53 and 51 in the manner described above with regards to fixation device 1. Specifically, rotation of anchors 200c and 200f relatively to one another creates a corresponding displacement of anchors 200d and 200g which is resisted by the rod segments interconnecting the aforementioned anchors. Rod 60 further includes rod segments 62, 63, 66, and 67 as shown in FIG. 4. Rod segments 62, 63 connect rod segment 57 with rod segments 58 and 52. Rod segment 66 includes rod segments 66a, 66b, and 66c, while rod segment 67 includes rod segments 67a, 67b, and 67c. Similar to rod 50 (FIG. 1), rod 60 is illustrated as including a plurality of rod segments for ease of explaining the disclosed features, it is contemplated that rod 60 may be a single continuous rod that is shaped to fit the desired anchor locations or may be a number of rod segments coupled together that form rod 60.

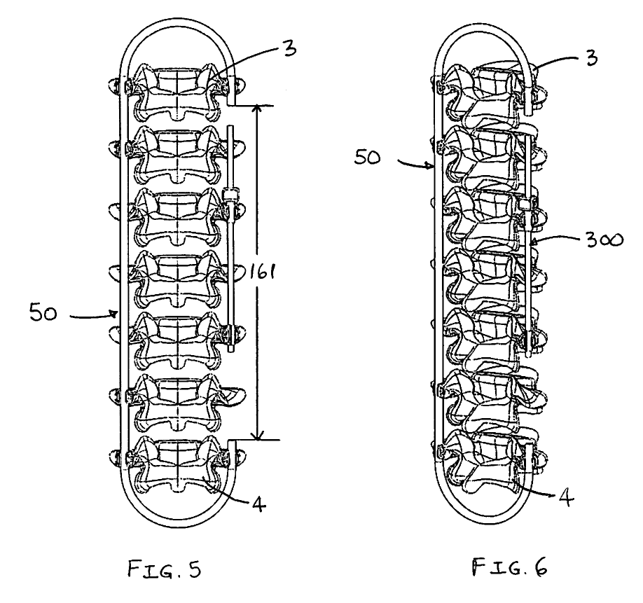

As shown in FIGS. 5 and 6, an additional stabilization device 300 such as a coupled rod device, a sliding rod device, and anchors may be disposed within corrective gap 161 without coming in contact with the anti-torsion spine fixation device. The additional stabilization device may include, for example, an automatically lengthening spine device such as that disclosed by commonly assigned PCT application PCT/US2009/33553 filed on Feb. 9, 2009, the disclosure of which is herein incorporated by reference in its entirety. Such devices are generally referred to as “growing spine devices.” Other known growing spine devices include, for example, distraction rods such as those disclosed by Bumpus in U.S. Pat. No. 4,931,055 and implantable spinal distraction splints such as those disclosed by Ulrich in U.S. Pat. No. 4,658,809.

The use and function of spinal fixation device 1 will be discussed during the course of a typical installation procedure and as part of the treatment of one or more scoliotic deformities. Initially, the location, orientation, and breadth of one or more scoliotic curves on a patient's spine will be determined using methods known in the art. Next, an operator identifies at least one caudad vertebrae 4 and cephalad vertebrae 3 for each curve such that a substantial portion of the curve is disposed between the aforementioned caudad and cephalad vertebrae. Next, an operator will secure at least two anchors to each selected vertebrae using methods commonly known in the art such that the anchors are disposed on opposing pedicles of their respective vertebrae.

A configuration of anchors and screws corresponding to the preceding paragraph is shown in FIGS. 1 and 2.

Next, in the event that only one pair of caudad and cephalad vertebrae have been selected, an operator will couple spinal fixation rod 50, 60 to each anchor using a set screw as shown respectively in FIGS. 1 and 3, a cam/clamp as is known in the art, or any other combination of rod coupling devices known in the art. Spinal fixation rod 50 includes a plurality of rod segments configured in a shape approximating a “C” such that the fixation rod spans the convex portion of the curve while there is no corresponding structure on the concave portion, defining a corrective opening. Further, the path of the rod segments defines arcuate caudad and cephalad rod portions which join the opposing anchors disposed on their respective caudad and cephalad vertebrae.

A configuration of anchors and fixation rod segments corresponding to the preceding paragraph is shown in FIGS. 1 and 2.

As a patient grows, the spacing of the vertebrae at the joined convex side of the scoliotic curve remains relatively constant, while the spacing of the vertebrae at the corrective gap corresponding to the convex portion is allowed to expand with the patient's growth. Further, the long segments of the spinal fixation rod provide improved torsional coupling for the device thereby reducing the tendency of the spine to develop new torsional deformities.

Finally, all or part of the device may be surgically removed or altered at the conclusion of modification of treatment.

It will be understood that various modifications may be made to the embodiments of the presently disclosed spinal fixation systems. Therefore, the above description should not be construed as limiting, but merely as exemplifications of embodiments. Those skilled in the art will envision other modifications within the scope and spirit of the present disclosure.

What is claimed is:

1. A spinal fixation device comprising:
a first anchor adapted to be attached to a first vertebra;
a second anchor adapted to be attached to the first vertebra,
a third anchor adapted to be attached to a second vertebra;
a first portion of a fixation member coupled to the first and second anchors, the first portion of the fixation member defining a path which approximates an arc between the first and second anchors; and
a second portion of the fixation member coupled to the second and third anchors.

2. The spinal fixation device of claim 1, wherein the first and second portions of the fixation member are coupled to their respective anchors by a set-screw.

3. The spinal fixation device of claim 1, wherein the first and second portions of the fixation member are coupled to their respective anchors by a taper lock mechanism.

4. The spinal fixation device of claim 1, wherein the anchors are disposed on the pedicles of the vertebrae to which they are attached.

5. The spinal fixation device of claim 1, wherein the anchors are pedicle screws.

6. A spinal fixation device comprising:
first and second anchors attachable to a first vertebra;
a third anchor attachable to a second vertebra;
a first portion of a fixation member extending from the first anchor to the second anchor, the first portion defining a first path; and
a second portion of the fixation member extending from the second anchor to the third anchor, the second portion defining a second path, wherein the first path and the second path define an angle therebetween.

7. The spinal fixation device of claim 6, wherein the angle is an acute angle.

8. The spinal fixation device of claim 6, wherein the fixation members are coupled to their respective anchors by a set-screw.

9. The spinal fixation device of claim 6, wherein the fixation members are coupled to their respective anchors by a taper lock mechanism.

10. The spinal fixation device of claim 6, wherein the anchors are disposed on the pedicles of the vertebrae to which they are attached.

11. The spinal fixation device of claim 6, wherein the anchors are pedicle screws.
